(19) United States
(12) Patent Application Publication
(10) Pub. No.: US 2025/0388648 A1
(43) Pub. Date: Dec. 25, 2025

(54) LOW MOLECULAR WEIGHT HYDROLYZED COLLAGEN COMPOSITION

(71) Applicant: GEORGE D. PETITO, Bethlehem, PA (US)

(72) Inventor: GEORGE D. PETITO, Bethlehem, PA (US)

(21) Appl. No.: 18/752,180

(22) Filed: Jun. 24, 2024

(51) Int. Cl. C07K 14/78; A61K 8/65; A61K 38/39; A61K 47/42; A61L 27/24; A61L 27/54; A61L 27/58; A61Q 19/08

(52) U.S. Cl. CPC C07K 14/78; A61K 8/65; A61K 38/39; A61K 47/42; A61L 27/24; A61L 27/54; A61L 27/58; A61Q 19/08

(57) ABSTRACT

A low molecular weight hydrolyzed collagen composition is provided. The low molecular weight hydrolyzed collagen composition is a medicinal composition for facilitating the growth, protection and healing of tissues and cells in animals and humans. The main ingredients of the formulated composition are hydrolyzed collagen having a molecular weight of less than 1,000 Da, which may be combined with one or more additives, such as polysulfated glycosaminoglycans, hyaluronic acid or salts thereof, or a glucosamine salt, and mixtures thereof. The composition may include gelatin. The composition may include low molecular weight collagen from multiple sources, such as bovine sourced collagen and/or marine sourced collagen and may further include hydrolyzed whey.

### BACKGROUND

### 1. Field

A low molecular weight hydrolyzed collagen composition and method of using the low molecular weight hydrolyzed collagen for medical, cosmetic, and nutritional purposes, and particularly, to a method and composition using low molecular weight hydrolyzed collagen.

### 2. Description of the Related Art

Just as nature has provided the skin as a barrier for protection, it has also provided mechanisms for skin repair. Depending upon the nature of the injury, this repair process may take hours, days, months, or even years. Many factors determine the length of time it takes for an injured skin to heal. Pathogenic contaminants may enter the body through the wound until the skin's integrity is restored. For this reason, it desirable to heal open wounds as quickly as possible.

Open wounds in the skin are a potential gateway for infectious or contaminating material to enter the body. The skin is a protective barrier to external contaminants. When the skin is damaged with an open breach, these contaminants are free to enter the body. Once inside the body, these contaminants may have effects of varying degrees, but almost always become more difficult to treat, and consequently slow the healing process of the original wound.

To fight infection, wound management traditionally involves an initial cleansing of the affected area to remove any contaminants such as dirt, clothing particles, or other debris. Damaged tissues and foreign materials are removed when necessary, and antiseptic agents are applied to sterilize the injured area. Sterile dressings are often applied, and periodically changed, to keep the injured area as clean and sterile as possible. Complex biological mechanisms occur during the healing process such as chemical signals attracting fibroblast cells to the wound site which ultimately generate connective structures mainly of collagen. Endothelial cells generate new blood capillaries that nurture new growth. The cell growth continues until the open wound is filled by forming permanent new tissue.

Because shortened periods of healing result in shortened exposure time, it would be beneficial to have any open wound heal as quickly as possible.

Traditional methods of wound healing have disadvantages, such as incomplete pigment removal, non-selective tissue destruction, and unsatisfactory cosmetic results, such as atrophic or hypertrophic scarring.

Thus, a wound healing composition and method solving the aforementioned problems is desired.

### SUMMARY

Low molecular weight collagen (“LMW collagen”), particularly collagen having a molecular weight of less than 1,000 Daltons or collagen having a molecular weight of less than 500 Daltons, may be useful in applications ranging from medical, to cosmetic, to physical and/or chemical carrier systems. LMW collagen may be formulated in a variety of physical configurations including but not limited to formulation as a liquid, solution, hydrogel, powder, freeze-dried powder, or as part of a porous scaffold or a microporous scaffold.

In an embodiment, the present subject matter relates to a composition composed of LMW collagen. The LMW collagen may act as a structural support/scaffold for diverse applications including acting as a space filler, a filling agent, presentation of adhesions, a carrying agent for drugs, a topical treatment, or for inclusion in cosmetic make-up products.

In an embodiment, the present subject matter relates to a composition composed of a mixture of LMW collagen and gelatin, which shall be referred to herein as “LMW-G”. The combination of LMW collagen and gelatin permits LMW-G to provide rapid bio intake to a tissue to which it is applied while acting as a structural support/scaffold for diverse applications including acting as a space filler, a filling agent, presentation of adhesions, a carrying agent for drugs, a topical treatment, or for inclusion in cosmetic make-up products.

Potential additives to LMW-G include most synthetic and semi-synthetic materials. For example, LMW-G may include glycosaminoglycans, polyvinyl alcohol (PVA), polypeptides, alginates, and naturally derived polymers (chitosan, fibrin, or the like), as well as any other suitable synthetic and semi-synthetic materials.

The LMW collagen can have increased bioavailability. The LMW collagen may comprise hydrolyzed collagen having a molecular weight of less than about 1,000 Da, less than about 500 Da, or less than about 400 Da. In an embodiment, the LMW collagen may comprise hydrolyzed collagen having a molecular weight ranging from about 10 Da to about 1,000 Da. For example, the LMW hydrolyzed collagen may comprise hydrolyzed collagen having a molecular weight of between about 1,000 Da and about 100 Da, between about 500 Da and about 100 Da, or between about 400 Da and about 100 Da. In a further embodiment, the LMW collagen may consist of hydrolyzed collagen having a molecular weight of less than about 1,000 Da, less than about 500 Da, or less than about 400 Da. In a further embodiment, the LMW collagen may consist of hydrolyzed collagen having a molecular weight of between about 1,000 Da and about 100 Da, between about 500 Da and about 100 Da, or between about 400 Da and about 100 Da.

In a further embodiment, the LMW collagen in the composition may comprise a mixture of bovine sourced collagen, marine sourced collagen, and hydrolyzed whey. The ratio of bovine sourced collagen: marine sourced collagen: whey may be about 7:2:1. Accordingly, the composition may include about 70% bovine sourced hydrolyzed collagen, about 20% marine sourced hydrolyzed collagen, about 10% bovine derived hydrolyzed whey. Optionally, this embodiment may include about 1% gelatin. In an alternative embodiment, the composition may comprise between about 1% and about 90% gelatin, with a 7:2:1 ratio of bovine sourced collagen: marine sourced collagen: whey making up the balance of the composition. In a further alternative embodiment, the composition may comprise between about 1% and about 90% gelatin, between about 1% and about 5% of one or more additives, and a 7:2:1 ratio of bovine sourced collagen: marine sourced collagen: whey making up the balance of the composition. This embodiment may further include chicken sourced collagen, either as a replacement for the marine sourced collagen or in addition to the marine sourced collagen.

In a further embodiment, the LMW collagen in the composition may comprise a mixture of bovine sourced collagen, chicken sourced collagen, and hydrolyzed whey. The ratio of bovine sourced collagen: chicken sourced collagen: whey may be about 7:2:1. Accordingly, the composition may include about 70% bovine sourced hydrolyzed collagen, about 20% chicken sourced hydrolyzed collagen, about 10% bovine derived hydrolyzed whey. Optionally, this embodiment may include about 1% gelatin. In an alternative embodiment, the composition may comprise between about 1% and about 90% gelatin, with a 7:2:1 ratio of bovine sourced collagen: chicken sourced collagen: whey making up the balance of the composition. In a further alternative embodiment, the composition may comprise between about 1% and about 90% gelatin, between about 1% and about 5% of one or more additives, and a 7:2:1 ratio of bovine sourced collagen: chicken sourced collagen: whey making up the balance of the composition. The addition of chicken sourced collagen as a substitute for marine sourced collagen, or the addition of the chicken sourced collagen may increase the ratio of Type 3 and Type 2 collagen with respect to the amount of Type 1 collagen. This ratio of collagen types may be adjusted to target specific types of wound care management, such as for full thickness wound management or implantable applications. These and other features of the present subject matter will become readily apparent upon further review of the following specification.

### DETAILED DESCRIPTION OF THE PREFERRED EMBODIMENTS

### Definitions

Throughout the application, where compositions are described as having, including, or comprising specific components, or where processes are described as having, including, or comprising specific process steps, it is contemplated that compositions of the present teachings can also consist essentially of, or consist of, the recited components, and that the processes of the present teachings can also consist essentially of, or consist of, the recited process steps.

It is noted that, as used in this specification and the appended claims, the singular forms “a”, “an”, and “the” include plural references unless the context clearly dictates otherwise.

In the application, where an element or component is said to be included in and/or selected from a list of recited elements or components, it should be understood that the element or component can be any one of the recited elements or components, or the element or component can be selected from a group consisting of two or more of the recited elements or components. Further, it should be understood that elements and/or features of a composition or a method described herein can be combined in a variety of ways without departing from the spirit and scope of the present teachings, whether explicit or implicit herein.

The use of the terms “include,” “includes”, “including,” “have,” “has,” or “having” should be generally understood as open-ended and non-limiting unless specifically stated otherwise.

The use of the singular herein includes the plural (and vice versa) unless specifically stated otherwise. In addition, where the use of the term “about” is before a quantitative value, the present teachings also include the specific quantitative value itself, unless specifically stated otherwise. As used herein, the term “about” refers to a +10% variation from the nominal value unless otherwise indicated or inferred.

The term “optional” or “optionally” means that the subsequently described event or circumstance may or may not occur, and that the description includes instances where said event or circumstance occurs and instances in which it does not.

Unless defined otherwise, all technical and scientific terms used herein have the same meaning as commonly understood to one of ordinary skill in the art to which the presently described subject matter pertains.

Where a range of values is provided, for example, concentration ranges, percentage ranges, or ratio ranges, it is understood that each intervening value, to the tenth of the unit of the lower limit, unless the context clearly dictates otherwise, between the upper and lower limit of that range and any other stated or intervening value in that stated range, is encompassed within the described subject matter. The upper and lower limits of these smaller ranges may independently be included in the smaller ranges, and such embodiments are also encompassed within the described subject matter, subject to any specifically excluded limit in the stated range. Where the stated range includes one or both of the limits, ranges excluding either or both of those included limits are also included in the described subject matter.

Throughout the application, descriptions of various embodiments use “comprising” language. However, it will be understood by one of skill in the art, that in some specific instances, an embodiment can alternatively be described using the language “consisting essentially of” or “consisting of”.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about”. Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

As used herein, “hydrolyzed collagen” is defined as a collagen hydrolysate polypeptide having a molecular weight lower than native collagen, i.e., in the 10 to 300,000 Daltons range, and is derived by hydrolysis.

As used herein, “hyaluronic acid” (HA) is rapidly hydrolyzed upon contact with treated tissue surfaces to monosaccharides, glucuronic acid, and N-acetyl glucosamine. Chemical binding is enhanced with the use of hydrolyzed collagen, i.e., it is chemotactic. Hyaluronic acid can be used via injection into a joint for its anti-inflammatory effect to relieve pain and suffering. This curative effect is inherently terminated when hyaluronic acid is consumed by the healing body.

As used herein, “glycosaminoglycans” (GAGs) are polysaccharides found in vertebrate and invertebrate animals. Several GAGs have been found in tissues and fluids of vertebrate animals. The known GAGs are chondroitin sulfate, keratin sulfate, dermatan sulfate, hyaluronic acid, heparin, and heparin sulfate. GAGs and collagen are the major structural elements of all animal tissue. Their synthesis is essential for proper repair, treatment, protection, and maintenance of all tissues.

As used herein, “chondroitin sulfate”, a polysulfated GAG, is a linear polymer occurring in several isomers, named for the location of the sulfate group. Chondroitin-4 sulfate is found in nasal and tracheal cartilages of bovines and porcine. It is also found in the bones, flesh, blood, skin, umbilical cord, and urine of these animals. Chondroitin-6 sulfate has been isolated from the skin, umbilical cord, and cardiac valves of the aforementioned animals. Chondroitin-6 sulfate has the same composition, but slightly different physical properties from the chondroitin-4 sulfate. These are the most common isomers used herein. The polymers are also known as polysulfated glycosaminoglycans (PSGAGs), chondroitin polysulfate sodium, chondrin, sodium chondroitin polysulfate, and sodium chondroitin sulfate. For consistency, the term, “chondroitin sulfate”, will be recited for all such chondroitin sulfate isomers, or any other chondroitin sulfate isomers, throughout this specification. Chondroitin sulfate is involved in the binding of collagen and is also directly involved in the retention of moisture in the tissue. These are both valuable chemical properties that aid the healing process.

As used herein, “subject” may refer to any animal, including but not limited to human beings and other mammals.

As used herein, “patient” may refer to a subject in need of treatment of a condition, disorder, or disease, such as an inflammatory condition or an immunological disorder.

As used herein, “Gelatin” refers to a collection of peptides and proteins produced by partial hydrolysis of collagen extracted from the skin, bones, and connective tissues of animals such as domesticated cattle, chicken, pigs, and fish. Generally, gelatin is understood to include (i) single α-chains of 80-125 kDa, (ii) two α-chains crosslinked in a covalent ay (β-chains) of 160-250 kDa,or (iii) three covalently crosslinked α-chains (γ-chains) of 240-375 kDa.

As used herein, “Bloom” refers to a measure of the gel strength of gelatin and relates to the molecular weight of its constituents. A higher bloom number is indicative of a “stiffer” gelatin. Generally, “low bloom” is understood to mean gelatin having a bloom number between 30-150, and reflects gelatin having an average molecular mass between 20,000-25,000; “medium bloom” is understood to mean gelatin having a bloom number between 150-225, and reflects gelatin having an average molecular mass between 40,000-50,000; and “high bloom” is understood to mean gelatin having a bloom number between 225-325 and reflects gelatin having an average molecular mass between 50,000-100,000.

### LMW-G

Low molecular weight collagen (“LMW collagen”), particularly collagen having a molecular weight of less than about 1,000 Daltons or collagen having a molecular weight of less than about 500 Daltons, may be useful in applications ranging from medical, to cosmetic, to physical and/or chemical carrier systems. LMW collagen may be formulated in a variety of physical configurations including but not limited to formulation as a liquid, solution, hydrogel, powder, freeze-dried powder, or as part of a porous scaffold or a microporous scaffold.

In an embodiment, the present subject matter relates to a composition composed of LMW collagen. The LMW collagen may act as a structural support/scaffold for diverse applications including acting as a space filler, a filling agent, presentation of adhesions, a carrying agent for drugs, a topical treatment, or for inclusion in cosmetic make-up products.

In an embodiment, the present subject matter relates to a composition composed of a mixture of LMW collagen and gelatin, which shall be referred to herein as “LMW-G”. The combination of LMW collagen and gelatin permits LMW-G to provide rapid bio intake to a tissue to which it is applied while acting as a structural support/scaffold for diverse applications including acting as a space filler, a filling agent, presentation of adhesions, a carrying agent for drugs, a topical treatment, or for inclusion in cosmetic make-up products.

A wide variety of additives may be incorporated into the LMW-G either separately or in combination with each other. Potential additives to LMW-G include most synthetic and semi-synthetic materials. For example, LMW-G may include glycosaminoglycans, polyvinyl alcohol (PVA), polypeptides, alginates, and naturally derived polymers (chitosan, fibrin, or the like), as well as any other suitable synthetic and semi-synthetic materials.

In an embodiment, the LMW-G may be formulated as a scaffold. This scaffold may be made in the form of a sheet or may be lyophilized into various sizes/types of solid cellular structures. These structures may be used as fillers, as supporting structures in surgery, for anti-adhesion properties, as nutrient chemical transmission, in collagen biosynthesis, as scaffolds for stem cell adhesion, as carriers for topical skin treatments, as cosmetics or wrinkle removers, or may be chemically bonded to separate raw materials requiring excellent absorption.

As used herein, the term “tissue engineering” is defined as application of the principles and methods of engineering and life sciences toward fundamental understanding of the structure-function relationship in normal and pathological mammalian tissues and the development of biological substitutes for the repair or regeneration of tissue or organ function.

Many issues exist in each product that is currently available to address the market sector discussed herein. The present disclosure relates to new compositions that address each such issue in the respective market sector. The LMW-G provides a functional support for the presently discussed compositions.

By way of non-limiting example, the LMW-G may be used in wound dressings, and depending upon the type of wound dressing (such as a powder, gel, solution, sheet, sponge, scaffold, or the like), the wound dressing formulation may start from the LMW-G. The LMW-G may be formulated in any of the wound dressing types discussed above. In one embodiment, the LMW-G may be formulated as a semi-permanent gel structure.

Potential market uses for the LMW-G include products having the LMW-G provided as a gel, sheet, or sponge. Tissue engineering applications for the LMW-G are possible for tissue and organ transplants. The LMW-G may be used as a delivery vehicle for a wide array of bioactive substances, including bioactive peptides, substances capable of attracting cells for tissue repair, and space fillers that could be used in organ transplants. When used in this capacity, the LMW-G may provide anti-adhesion benefits, and may be used in applications targeting bone, cartilage, and muscle mass.

The gelatin component of the LMW-G can be selected for specific “bloom” characteristics, which may allow the specific composition to be time-released and targeted to filling a specific space requirement of a particular application.

The LMW-G may be formulated using cross-linking for additional advantages. Cross-linking may be achieved using either chemical treatments or during sterilization, such as by sterilizing using gamma radiation. In one embodiment, stable cross-linking of LMW-G may be achieved by exposure to gamma radiation at about 60 to about 80 kGy. This treatment produces both improved stability and moderate cross linking of the final product, thus making it suitable as a carrier, filler, and biodegradable when applied within the body.

In certain embodiments, where timed release is of importance, native collagen may be incorporated into the LMW-G to delay the natural rate of degradation/absorption of the LMW-G, thereby delaying the release of any active agents that may be incorporated therein.

In certain embodiments, the LMW-G may serve as a scaffold, particularly for use in treating bone defects. When used in this context, the LMW-G can be advantageous for bone regeneration by providing nutrient support and promoting cell growth.

In certain embodiments, the LMW-G may be formulated for use with tissue engineering applications. In this context, the LMW-G may provide highly biosorption factors for attachment, may provide a framework for cell production/scaffolding, and may provide chemical attachment with chemotactic properties.

The water vapor transmission rate (WVTR) is generally determined for films by measuring the quantity of water vapor that passes through the film during a fixed time. In one embodiment, LMW-G may be provided in powder form and made into a hydrogel for testing, or the LMW-G powder may be subjected to testing using a film with a known WVTR, by adding the LMW-G powder to the film and retesting the film. In a further non-limiting example, a LMW-G gel can be cast upon a known film, and then tested. For example, a topical wound dressing having a WVTR less than 35 g/m³ is defined as moisture retentive. Wounds are known to heal faster when moisture levels are controlled. The physical configuration of a hydrogel will confer its mechanical properties. As noted previously, by using minimal cross-linking of the LMW-G gel, one can produce a scaffold/matrix that is much more biodegradable than when using heavily cross-linked LWG-G. Heavily cross-linked LMW-G has a greater density and is stronger than minimally cross-linked LMW-G, and thus is particularly useful for applications such as ligament and tendon repair.

The addition of gelatin in the LMW-G can provide improved rheological properties to the end product. By selecting gelatin with a particular bloom, mixing gelatins with different blooms, and controlling the amount of cross-linking, one can determine the shear viscosity of the final product. This allows the final product to be tailored to specific desirable properties for treatment. For example, when formulated for tendon repair, the LMW-G will typically use gelatin having a higher bloom. Wound treatment products may be formulated depending upon the specific wound healing stage, such as hemostasis, inflammatory, proliferative, or maturation. For example, LMW-G may be formulated to control bleeding (hemostasis) with the gelatin bloom adjusted for speed, hemostasis and biodegradability. In an embodiment, the LMW-G formulated for this particular purpose may have a bloom of at least 120.

In a further embodiment, LMW-G may be used to treat adhesive lesions. In this embodiment, the product can be adjusted for inducing a response to inflammation at a speed that has not previously been demonstrated. This allows the product to “jump start the wound site”. The use of LMW-G in this formulation will increase the inflammatory response for a short period of time (generally 12 to 24 hours), but will not exceed the wound's threshold. During this stage the damaged cells, bacteria, and the like are typically removed. This lays the foundation for new tissue to contract the wound, and to form granulation tissue more rapidly. In this embodiment the LMW-G is provided in gel form as a hydrogel with the gelatin bloom selected for slow dissipation (i.e., higher bloom gelatin is selected).

During the final stage of wound healing and maturation, collagen is remodeled from type 3 to type 1 and the wound is closed. At this stage LMW-G can provide numerous advantages. By way of non-limiting example, the use of LMW-G may result in the wound strength, which is generally understood to be weaker when just healed than the surrounding skin, to actually be equal to or stronger than the surrounding skin. Further, the use of LMW-G can shorten the amount of time required for a wound site's strength to return to equal or greater than the surrounding skin. The specific use of LMW collagen in the LMW-G can provide higher bioavailability, faster biodegradability, and chemical/physical alterations such as reduced dissolving time. The inclusion of the LMW collagen in the LMW-G may act in part by providing decreased absorption or increased viscosity.

In some embodiments, the LMW-G can be crosslinked to one or more active agents, which may be selected based upon a specific desired application.

In some embodiments, the LMW-G may remain un-crosslinked, for applications where speed of dissolution in the wound site is desirable. In these embodiments, the LMW-G may also act as a carrier, provided rapid delivery of specific active agents useful for wound healing. For example, glycosaminoglycans such as chondroitin sulfate may be added to the LMW-G, in applications where denser granulation tissue is desired. This example may be particularly useful for tendon repair, and the like. In a further specific embodiment, bioactive glass may be added to the LMW-G, to adjust the rheological properties of the final product and further enable faster active delivery to the tissue site.

The LMW-G compositions disclosed herein may be formulated for a broad range of uses, including both medical applications (including but not limited to formulations for wound healing, oral medical foods, and the like) and other applications such as cosmetic and industrial applications. Cosmetic applications of the LMW-G can also take advantage of its unique advantages with respect to acting as a carrier and its capability of delivering various chemicals in a controlled manner. The LMW-G as described herein uses very low molecular weight collagen, which results in a composition that has an improved ability to penetrate through the skin and provide increased bioavailability of any active agents carried by the LMW-G, as well as of the LMW-G components themselves. Thus, LMW-G may be used in wrinkle removal, scar treatment, and acne preparations, and the like.

Similarly, specific medical applications such as use post skin abrasion, post operative dermatological treatment, and treatment of ulcers may be particularly advantaged by the carrier delivery capabilities of the present LMW-G. In a particular embodiment, the LMW-G may be useful in treating pre-dermal abraded skin, thereby preventing an ulcer from actually opening.

When formulating LMW-G for topical wound treatment, the gelatin can comprise between 1% and 90% of the final compound. Considerations that may impact gelatin concentration include the wound type, whether the wound is acute or chronic, the depth and size of the wound and the morbidity factors of the patient. The selected concentration of the gelatin, as well as the bloom of the specific gelatin used will both be directly related the length of stay of the final compound, the absorbance, and the dressing change intervals.

When formulating LMW-G for topical applications for use on unabraded skin the amount of gelatin used will be much less than when used for open wound healing. In an example, these formulations may include between about 1% and about 10% gelatin. In these formulations the amount of gelatin used will be sufficient to hold drying times and support desired applications. This formulation may use one or more additives, such as calamine, vitamins, active agents known for acne control activity, sunscreens, active agents known for scar control activity, and the like.

When formulating LMW-G for oral administration, the concentration of gelatin can comprise between about 1% and about 50% of compositions intended for use as nutritional supplements, or from about 5% to about 35% of compositions intended for use as medical foods. These formulations may also include one or more additives that may be tailored to a particular intended use. For example, when the LMW-G is intended for joint support, the additives may include one or more of chondroitin sulfate, glucosamine sulfate or hydrochloride, Omega-3, DHA, and sodium hyaluronate. When the LMW-G is intended for use by athletes, the additives may include one or more of glycosaminoglycans, wheat starch, wheat isolates, albumin, nano-formulated proteins (of both animal and vegan origin), pectin, chitosan, chitin, cellulose, and the like. When the LMW-G is intended for use as a medical food, the additives may include one or more of Omega-3, zinc, alpha lipoic acid, B complex vitamins, other vitamins, L-citrulline, berry extracts, glutamine, collagen, hydrolyzed collagen, collagen isolates, essential amino acids, non-essential amino acids, glymnema sylvestre, white mulberry, and the like.

It is noted that the use of additives with the LMW-G can result in significant levels of increased bioactivity when compared to other known formulations. Further, the additives may be chemotactically attached to the LMW-G.

Hydrolyzed collagen is a collagen hydrolysate polypeptide having a molecular weight lower than native collagen. Hydrolyzed collagen may be obtained by hydrolysis of native collagen. This may be accomplished by one of four methods: (1) alkaline hydrolysis; (2) enzymatic hydrolysis; (3) acid hydrolysis; and (4) synthetically, by fermentation. Any of these methods can be used to derive the hydrolyzed collagen from a collagen source.

The native collagen and the hydrolyzed collagen can be derived from any suitable collagen source. The collagen source can be, for example, a bovine (skin and tendon preferred), a porcine, a reptile, a marine, an avian, a vegan, or a synthetic source. The collagen can be derived from a single collagen source or a combination of collagen sources. The marine source can include any fish. The types of amino acid constituents and their sequences determine the beneficial healing qualities of hydrolyzed collagen. Hydroxylysine and hydroxyproline are amino acids found only in collagen and in no other medical protein hydrolysates. Hydroxylysine is typically found in concentrations from 0.7 to 1.2 wt. % in hydrolyzed collagen.

Bovine and porcine hydrolyzed collagen have high glycine, proline, hydroxyproline, and glutamic acid content. They also display hydrophilic properties. Bovine hydrolyzed collagen, for example, demonstrates strong hydrophilic properties and when used to treat wound sites demonstrates increased perfusion and epithelialization and decreased inflammatory reaction. In contrast, marine derived, i.e., marine sourced, hydrolyzed collagen has a different amino acid profile, with higher levels of aspartic acid, cysteine, glutamine, citrulline, and asparagine.

The LMW collagen can have increased bioavailability. The LMW collagen may comprise hydrolyzed collagen having a molecular weight of less than 1,000 Da, less than 500 Da, or less than 400 Da. In an embodiment, the LMW collagen may comprise hydrolyzed collagen having a molecular weight ranging from 10 Da to 1,000 Da. For example, the LMW hydrolyzed collagen may comprise hydrolyzed collagen having a molecular weight of between 1,000 Da and 100 Da, between 500 Da and 100 Da, or between 400 Da and 100 Da. In a further embodiment, the LMW collagen may consist of hydrolyzed collagen having a molecular weight of less than 1,000 Da, less than 500 Da, or less than 400 Da. In a further embodiment, the LMW collagen may consist of hydrolyzed collagen having a molecular weight of between 1,000 Da and 100 Da, between 500 Da and 100 Da, or between 400 Da and 100 Da.

The LMW collagen can be prepared by partially hydrolyzing native collagen in any suitable manner known in the art. In one embodiment, raw materials from one or more collagen sources are ground to a powder, enzymatically treated, fractionated, and purified to obtain high molecular weight hydrolyzed collagen. Bulk fractionation methods known in the art can be used. The raw materials can include, for example, fat, blood, tissue, and/or bone marrow from one or more collagen sources. Raw material from fish can further include, e.g., fish head and/or fins.

The LMW-G composition may include about 1% by weight to about 99% by weight LMW collagen. For example, the composition may include about 10% by weight to about 85% by weight LMW collagen or about 20% by weight to about 75% by weight LMW collagen, or about 30% by weight to about 65% by weight LMW collagen. In an embodiment, the LMW collagen can be LMW hydrolyzed collagen. The LMW-G composition may further include about 1% to about 90% gelatin.

In an embodiment, the composition can include mixtures of collagen from different collagen sources. For example, the composition can include bovine sourced collagen, marine sourced collagen, chicken sourced collagen, and whey protein. Alternatively, the composition can include bovine sourced collagen and marine sourced collagen. According to one embodiment, the proteinaceous amino acids in the composition can include bovine sourced hydrolyzed collagen, marine sourced hydrolyzed collagen, and hydrolyzed whey protein. The composition can further include elastin. Hydrolyzed whey protein offers another alternative amino acid profile, rich in glutamic acid, isoleucine, leucine, threonine, tyrosine, and valine.

Varying the source of the amino acids in the composition can control the chemotactic, hydrophilic, and cell proliferative properties of the composition. These properties may be manipulated to optimize the wound healing process. This optimization may adjust the timing and balance of stimulating the inflammatory and vascular systems, as well as involvement of connective tissues and epithelial cells.

For example, a heavily exudative or wet wound can be treated, at least initially, with a highly hydrophilic composition, including at least about 50% by weight bovine sourced hydrolyzed collagen (e.g., about 50% to about 70% by weight bovine sourced hydrolyzed collagen), at least about 20% by weight marine sourced hydrolyzed collagen (e.g., about 20% to about 30% by weight marine sourced hydrolyzed collagen), and up to about 30% by weight hydrolyzed whey protein (e.g., about 5% to about 30% by weight hydrolyzed whey protein). Soluble elastin may be added to this composition (e.g., up to about 20%) during the closing phase of wound treatment to improve tensile strength and reduce scar formation. In contrast, a dry wound can be treated with at least about 50% by weight marine sourced hydrolyzed collagen, about 20% by weight or less hydrolyzed whey, about 10% by weight or less bovine sourced hydrolyzed collagen, and about 5% by weight or less elastin.

In a further embodiment, the LMW collagen in the composition may comprise a mixture of bovine sourced collagen, marine sourced collagen, and hydrolyzed whey. The ratio of bovine sourced collagen: marine sourced collagen: whey may be about 7:2:1. Accordingly, the composition may include about 70% bovine sourced hydrolyzed collagen, about 20% marine sourced hydrolyzed collagen, about 10% bovine derived hydrolyzed whey. Optionally, this embodiment may include about 1% gelatin. In an alternative embodiment, the composition may comprise between about 1% and about 90% gelatin, with a 7:2:1 ratio of bovine sourced collagen: marine sourced collagen: whey making up the balance of the composition. In a further alternative embodiment, the composition may comprise between about 1% and about 90% gelatin, between about 1% and about 5% of one or more additives, and a 7:2:1 ratio of bovine sourced collagen: marine sourced collagen: whey making up the balance of the composition. This embodiment may further include chicken sourced collagen, either as a replacement for the marine sourced collagen or in addition to the marine sourced collagen.

In a further embodiment, the LMW collagen in the composition may comprise a mixture of bovine sourced collagen, chicken sourced collagen, and hydrolyzed whey. The ratio of bovine sourced collagen: chicken sourced collagen: whey may be about 7:2:1. Accordingly, the composition may include about 70% bovine sourced hydrolyzed collagen, about 20% chicken sourced hydrolyzed collagen, about 10% bovine derived hydrolyzed whey. Optionally, this embodiment may include about 1% gelatin. In an alternative embodiment, the composition may comprise between about 1% and about 90% gelatin, with a 7:2:1 ratio of bovine sourced collagen: chicken sourced collagen: whey making up the balance of the composition. In a further alternative embodiment, the composition may comprise between about 1% and about 90% gelatin, between about 1% and about 5% of one or more additives, and a 7:2:1 ratio of bovine sourced collagen: chicken sourced collagen: whey making up the balance of the composition. The addition of chicken sourced collagen as a substitute for marine sourced collagen, or the addition of the chicken sourced collagen may increase the ratio of Type 3 and Type 2 collagen with respect to the amount of Type 1 collagen. This ratio of collagen types may be adjusted to target specific types of wound care management, such as for full thickness wound management or implantable applications.

In a further embodiment, the LMW collagen in the composition may comprise a mixture of bovine sourced collagen, marine sourced collagen, and hydrolyzed whey. The bovine sourced collagen may be between about 20% and about 80% of the composition by weight, the marine sourced collagen may be between about 10% and about 80% of the composition by weight, and the whey may be between about 10% and about 50% of the composition by weight.

The additives may include one or more additional therapeutic agents, which by inclusion in the composition further speed the healing process and/or decrease scarring and increase tissue strength. Examples of suitable therapeutic agents that may be combined with the LMW-G are glycosaminoglycans (GAGs), particularly GAGs useful for cellular repair. Antimicrobials may also be included in the composition to further enhance its bacteriostatic quality, as can antibiotics (such as tetracycline, streptomycin, and cephalosporin) and antibacterials (such as iodine, parachlorometaxylenol, and chlorhexidine gluconate or acetate). The composition may further include lipoic acid, alpha lipoic acid, one or more vitamins (e.g., vitamin A, vitamin B12, vitamin C, vitamin E), omega compounds or omega-3 fatty acid compounds (e.g., α-linolenic acid “ALA”, Eicosapentaenoic acid “EPA”, Docosahexaenoic acid “DHA”),), antioxidants (e.g., superoxide dismustase, glutathione peroxidase, glutathione reductase), and/or phytochemicals (e.g., zeaxanthin, lutein). Also, it has been established that hydrolyzed collagen used as a carrier in powder form, paste or a lyophilized foam has hemostatic qualities when combined with thrombin to improve healing of wounds.

LMW-G in combination with GAGs, specifically a PSGAG (such as chondroitin sulfate), can be useful for the prevention and treatment of wound diseases. The hydrolyzed collagen can be combined with a PSGAG to bond or adhere selectively to tissue, resulting in interference with and/or displacement of bacterial or other infectious agents. In addition, the combination product may exhibit anti-enzyme activity or the ability to inhibit enzyme activity.

LMW-G can accelerate the healing process by allowing an injured tissue to repair itself by producing and remodeling more collagen and other proteoglycans (PGs). The building blocks for collagen production are the amino acids found in hydrolyzed collagen. Hyaluronic acid and other proteoglycans (PGs) can provide the framework for collagen production to follow. The PGs can hold water to provide an excellent environment for healing of the tissue to begin. When in the wound site, any unused collagen that was produced is typically simply degraded to amino acids. The rate-limiting step in the production of collagen is typically the conversion of glucose to glucosamine for the production of hyaluronic acid and other glycosaminoglycans (GAGs).

The present compositions can include one or more therapeutic agents, such as an antibiotic, and/or one or more additives, such as glutamine, glycosaminoglycans, zinc, alginates, cellulose, bioactive glass, and/or honey.

These are simplified examples, as wound healing is complex and wound specific. More complicated wounds, such as diabetic wounds, are treated using customized treatment regimens. For example, a diabetic wound can initially be treated as a wet wound, but with significantly more emphasis on hydrolyzed whey in the early treatment composition. During the later closing phase of wound treatment, the composition can be shifted to up to about 40% by weight marine sourced collagen, up to about 25% by weight bovine sourced collagen, and up to about 5% by weight elastin.

Further examples of this wound healing composition optimized for different applications include: about 70% bovine sourced hydrolyzed collagen and about 30% marine sourced hydrolyzed collagen; about 50% bovine sourced hydrolyzed collagen, about 30% marine sourced hydrolyzed collagen, and about 20% hydrolyzed whey; about 40% bovine sourced hydrolyzed collagen, about 20% marine sourced hydrolyzed collagen, about 20% hydrolyzed whey, and about 20% elastin; about 20% bovine sourced hydrolyzed collagen, about 40% marine sourced hydrolyzed collagen, about 20% hydrolyzed whey, and about 20% elastin; about 20% bovine sourced hydrolyzed collagen, about 40% marine sourced hydrolyzed collagen, and about 40% hydrolyzed whey; and about 30% bovine sourced hydrolyzed collagen, about 30% marine sourced hydrolyzed collagen, about 30% hydrolyzed whey, and about 10% elastin. The elastin can have a molecular weight of about 35,000 Daltons to about 145,000 Daltons.

The present compositions may be used to heal topical and/or internal wound sites. For example, the present compositions may be used prior to and after surgery to minimize cell damage and to expedite wound healing. The present compositions may be useful during surgery to foster separation of tissue to prevent adhesion formation. The compositions may be used as a filler for a wound site and remain in the wound site as it heals, becoming part of the granulated tissue.

The present compositions may be useful for applications relating to cosmetic and plastic surgery, e.g., as a filler for lines and wrinkles formed in the skin.

The present compositions may take a physical form used in topical administration, such as a gel, spray, powder, paste, foam, film for incorporation in a dressing bandage, or a topically applied patch. The compositions may take a physical form used in internal administration, such as an injectable liquid or an orally ingestible liquid.

The present compositions may be formulated for use as a medical food. Medical foods are foods that are formulated to be consumed or administered under supervision of a physician and which are intended for the specific dietary management of a disease condition for which distinctive nutritional requirements are established by medical evaluation. The composition formulated for use as a medical food may be formulated for oral consumption or for tube feeding.

When formulated as a powder, the present compositions can have a moisture content of about 2-10 wt. % and a pH range of 5.5 to 6.5. The powder composition can have an ash content of less than about 2.5 wt. % and an isotonic point of about 5.0 to about 6.5. In use, the powder composition may be the preferred physical form for use with irregularly shaped wounds. Tunnel wounds, flaps, and other non-conformative sites may be managed with the powder composition because it easily conforms to any shape wound and may be applied by a poofer bottle or otherwise blown into difficult to reach wound sites. The powder is especially useful in wounds having a large amount of exudate, as the powder can absorb nearly 30 times its own weight. As the powder absorbs the exudate, a gel is formed, which completely fills the wound site, forming a mechanical barrier against bacterial infection. The powder does not exhibit the characteristic fly-away when being applied to the wound site, and administration is perfected due to the precise powder placement.

When formulated as a gel, the present compositions are especially useful in wounds with lesser amounts of exudate, in burns, and in surgical sites. Application of the gel can be dispensed through a tube, a syringe, or the reservoir in a topical patch. The gel can be made of about 1-75 wt. % hydrolyzed collagen and 1-99 wt. % water. It is preferable to use about 60 wt. % collagen. The gel is formed by adding purified water, e.g., sterile water, to the powder. The gel has the added advantage of adding moisture to the wound site, as well as inherent bacteriostatic properties, and stays positioned where applied.

When formulated as a film, the present compositions may be made by mixing the powdered form with deionized water under heat at 155-175° F. Cross-linking and other agents, such as humectant, propylene glycol, sorbitol, and glycerin, may be added to the mixture. A preservative (such as benzyl alcohol or paraben) can be added. The mixture can be cast on a belt liner by knife on a roll coating machine to form a liquid film, which can be oven dried. The film form can also be formed by cooling the liquid solution. These films can be used for drug or other chemical delivery, especially in dental applications. Antimicrobial and other medicinal agents can also be added to the film as needed for specific applications.

The present compositions may be formulated as a nutritional supplement. In an embodiment, the nutritional supplement can include at least suitable nutritional additive. For example, at least one of vitamin A, vitamin C, vitamin E, vitamin B12, magnesium oxide, chelated manganese, grape seed extract, zinc, an alginate, cellulose, honey, chromium picolinate, selenium, glutamine, alpha lipoic acid, Coenzyme Q10, and glycosaminoglycans can be added to the composition to produce a nutrient composition for oral intake.

The present compositions may also be administered as compositions prepared as foods for humans or animals, including medical foods, functional foods, special nutrition foods and dietary supplements. A “medical food” is a product prescribed by a physician that is intended for the specific dietary management of a disorder or health condition for which distinctive nutritional requirements exist and may include formulations fed through a feeding tube (referred to as enteral administration or gavage administration) or via intravenous injection (i.v.).

These medical foods may be formulated according to any method known in the art. For example, methods of formulating medical foods are discussed in U.S. Pat. Nos. 5,637,324 A and 6,210,701 B1, the contents of which are incorporated herein by reference.

A “dietary supplement” shall mean a product that is intended to supplement the human diet and may be provided in the form of a pill, capsule, tablet, or like formulation. By way of non-limiting example, a dietary supplement may include one or more of the following dietary ingredients: vitamins, minerals, herbs, botanicals, amino acids, and dietary substances intended to supplement the diet by increasing total dietary intake, or a concentrate, metabolite, constituent, extract, or combinations of these ingredients, not intended as a conventional food or as the sole item of a meal or diet. Dietary supplements may also be incorporated into foodstuffs, such as functional foods designed to promote control of glucose levels. A “functional food” is an ordinary food that has one or more components or ingredients incorporated into it to give a specific medical or physiological benefit, other than a purely nutritional effect. “Special nutrition food” means ingredients designed for a particular diet related to conditions or to support treatment of nutritional deficiencies.

When formulated as a dietary supplement, in addition to collagen the present compositions may include one or more of a vitamin, a glycosaminoglycan, alpha lipoic acid, a berry extract, and berberine. It is specifically contemplated that the dietary supplements disclosed herein may use liposomes as a delivery vehicle. In the alternative, the present compositions may include one or more polymeric molecules and or a biopolymer. In certain embodiments the present compositions may be formulated as films or as film-forming compositions.

The present compositions may be formulated for topical cosmetic applications, such as for anti-aging or anti-wrinkle applications. In such applications the compositions may include further ingredients, including one or more of an emulsifier, preservative, thickener, emollient, pigment, glimmer, fragrance, paraben, or a combination thereof. These compositions may be formulated by techniques generally known in the art, including those discussed in U.S. Pat. Nos. 10,905,640 B2, 6,524,459 B2, and US 2002/0058053 A1, the contents of which are incorporated herein by reference.

The present compositions can be used as an excellent drug vehicle system including acidic, neutral, or complexed drug medications.

### Methods of Wound Healing

The method of treating a wound may include administering at least one of the compositions disclosed above to a subject in need thereof.

In an embodiment, subjects in need thereof may be administered a composition including LMW bovine sourced collagen, LMW marine sourced collagen, and/or hydrolyzed whey. In certain embodiments, the composition may include about 70% LMW bovine sourced collagen, about 20% LMW marine sourced collagen, about 10% bovine derived hydrolyzed whey, and about 1% gelatin.

In a further embodiment, subjects in need thereof may be administered a composition including between about 1% gelatin and about 90%, the balance being made up of an about 7:2:1 ratio of LMW bovine sourced collagen, LMW marine sourced collagen, and hydrolyzed whey.

### Other Medical Uses

In some embodiments, the compositions disclosed herein may be used in combination with other active agents, including drugs, vitamins, glucosamine, glycosaminoglycans, other collagens, honey, aloe, and anesthetic agents including benzocaine, lidocaine, or the like. When used in a combination treatment, the collagen composition may act as a carrier and may improve the bioavailability, absorption, stability, or skin penetration of the other active agent.

In further embodiments, the compositions disclosed herein may be used as part of a filler treatment, including but not limited to use as a line filler restorer, or in compositions assisting in reducing the healing time of surgical closures or other internal wounds.

In some embodiments, the compositions disclosed herein may be used as soluble collagen injections, solid constructs reconstituted from solution, decellularized collagen matrices, or for treatment of chronic wounds, burns, venous or diabetic ulcers, or in plastic, reconstructive, cardiovascular, bone, cartilage, or general surgery, or generally in the practice of urology, proctology, gynecology, ophthalmology, otolaryngology, neurosurgery, dentistry, or cosmetology.

In some embodiments, the compositions disclosed herein may be used for cosmetic applications. The compositions disclosed herein may be useful additives in products intended to improve moisture retention, reduce the appearance of wrinkles, or the like. Further, the compositions disclosed herein may be formulated as topical cosmetic treatments (creams and the like), or as injectable compositions (for treatment of wrinkles and the like). When formulated for cosmetic applications, the compositions disclosed herein may further act as a carrier for other active agents, including but not limited to hyaluronic acid and the like.

It is to be understood that the low molecular weight hydrolyzed collagen composition compositions and methods are not limited to the specific embodiments described above, but encompasses any and all embodiments within the scope of the generic language of the following claims enabled by the embodiments described herein, or otherwise shown in the drawings or described above in terms sufficient to enable one of ordinary skill in the art to make and use the claimed subject matter.

We claim:

1. A composition comprising low molecular weight hydrolyzed collagen, wherein the low molecular weight hydrolyzed collagen has a molecular weight of less than 1,000 Daltons.

2. The composition of claim 1, wherein the low molecular weight hydrolyzed collagen comprises bovine derived collagen, marine derived collagen, and hydrolyzed whey in a ratio of 7:2:1.

3. The composition of claim 2, wherein the low molecular weight hydrolyzed collagen comprises about 70% bovine derived hydrolyzed collagen, about 20% marine sourced hydrolyzed collagen, and about 10% hydrolyzed whey.

4. The composition of claim 3, comprising chicken sourced collagen.

5. The composition of claim 2, wherein the low molecular weight hydrolyzed collagen comprises about 70% bovine derived hydrolyzed collagen, about 20% chicken sourced hydrolyzed collagen, and about 10% hydrolyzed whey.

6. The composition of claim 1, comprising about 1% to about 90% of the gelatin.

7. The composition of claim 6, wherein the gelatin has a bloom number between 30 and 150.

8. The composition of claim 6, wherein the gelatin has a bloom number between 150 and 225.

9. The composition of claim 6, wherein the gelatin has a bloom number between 225 and 325.

10. The composition of claim 1, further comprising at least one therapeutic agent selected from the group consisting of glutamine, glycosaminoglycans, zinc, alginates, cellulose, bioactive glass, honey, and a combination thereof.

11. A medical food comprising the composition of claim 1.

12. A nutritional supplement comprising the composition of claim 1.

13. A cosmetic comprising the composition of claim 1.

14. The composition of claim 1, further comprising an active agent selected from the group consisting of drugs, vitamins, glucosamine, glycosaminoglycans, native collagen, honey, aloe, benzocaine, lidocaine, and a combination thereof.

15. A method of treating a wound in a subject comprising administering the composition of claim 1 to a subject in need thereof.

16. A method of delivering the active agent to a subject in need thereof, comprising administering the composition of claim 1 to the subject.

17. A method of reducing wrinkles in a subject, comprising topical administration of the composition of claim 1 to a subject in need thereof.

18. A method of reducing photoaging of skin in a subject, comprising topical administration of the composition of claim 1 to a subject in need thereof.

19. A method of healing internal wounds in a subject, comprising administering the composition of claim 1 to a subject in need thereof.

20. The method of claim 19, wherein the method further comprises reducing the recovery time from a surgical procedure.
